(19) United States
(12) Patent Application Publication
(10) Pub. No.: US 2003/0163158 A1
(43) Pub. Date: Aug. 28, 2003

(54) Method and apparatus for performing percutaneous thromboembolectomies

(76) Inventor: Geoffrey H. White, New South Wales (AU)

Correspondence Address: NIXON & VANDERHYE, PC, 1100 N GLEBE ROAD, 8TH FLOOR, ARLINGTON, VA, 22201-4714, US

(21) Appl. No.: 10/311,232

(22) PCT Filed: Jun. 21, 2001
(86) PCT No.: PCT/AU01/00738

(30) Foreign Application Priority Data
Jun. 22, 2000 (AU) PQ 8315

(51) Int. Cl. A61M 29/00

(52) U.S. Cl. 606/200

(57) ABSTRACT

A thromboembolectomy device for performing a thromboembolectomy procedure in a bodily vessel. The device includes a thromboembolectomy means (30) capable of being moved relatively through the vessel and further capable of dislodging a thrombus from a wall of the vessel. A capture means (10) is positioned distal the thromboembolectomy means and is movable between a first collapsed configuration and a second expanded configuration. An extractor means (20) is further positioned proximal the thromboembolectomy means (30). The extractor means (20) has a lumen, a first end and a second end wherein at least the second end is capable of moving between a first collapsed state and a second expanded state. On dislodgment ol the thrombus from the vessel wall by the thromboembolectomy means (30), the dislodged thrombus can be drawn relatively towards the extractor means (20) for removal from the vessel and wherein further, any portions of thrombus that detach from the thrombus are retained by the capture means (10).

### FIELD OF THE INVENTION

The present invention relates to a method and apparatus for use in surgical procedures involving the removal of a thrombus or embolus from a vessel in the body of a patient, and more specifically, to percutaneous thromboembolectomies.

### BACKGROUND OF THE INVENTION

While thromboembolectomies have recently been performed percutaneously, it is still common practice to undertake removal of a thrombus or embolus by open surgery. During an open thromboembolectomy, the vessel from which a thrombus is to be removed is clamped and incised. Any one of a variety of thromboembolectomy means may then be used to remove the thrombus or embolus by, for example, scraping it from the wall of the vessel. Finally, the open ends of the vessel are re-anastomosed and the surgical wound is closed.

In circumstances where it is desirable to maintain blood flow through the vessel during the procedure, an open technique of performing the thromboembolectomy will not be appropriate. In such circumstances, a percutaneous method is preferred.

Percutaneous thromboembolectomies classically involve the use of a thromboembolectomy device or pharmacological substance which is capable of actual physical removal of the thrombus or embolus from the vessel. In this regard, a variety of approaches have been employed in the performance of such procedures. These include the removal of a thrombus or embolus via 25 suction during angioplasty, removal using various mechanical thrombolytic devices and drug induced thrombolysis with pharmacological substances such as urokinase.

Another approach involves the use of a balloon catheter such as that disclosed by Fogarty (U.S. Pat. No. 4,271,839). Removal of the thrombus is achieved by introducing a percutaneous sheath into a vessel, introducing a deflated or collapsed thromboembolectomy means into the vessel through the percutaneous sheath, extending it to a point distal to the thrombus, then expanding or inflating the thromboembolectomy means. The thromboembolectomy means is subsequently withdrawn from the vessel so scraping the thrombus from the vessel wall and drawing it out of the patient's body. The inherent difficulties which arise as a result of performing a percutaneous thromboembolectomy in this manner are manifold and include the fact that the thrombus, having been scraped from the vessel wall, will be in a traumatised state, loosely composed and, therefore, prone to break up. Drawing out a thrombus in such a state from the vessel through a percutaneous sheath carries with it a risk that not all of the thrombus will be removed. This may be due to the fact that, among other reasons, some of the thrombus is prevented from being removed by the distal edge of the percutaneous sheath or a narrow portion thereof.

A further difficulty arising from the procedure is that small portions from the surface of the thrombus may be dislodged and may embolise to more distal locations. Since the diameter of vessels tend to become smaller as they branch out and extend into the peripheries, this could have very serious consequences: the thromboembolus may, for example, completely occlude a smaller vessel.

The present invention is aimed at overcoming these, and other, difficulties as well as substantially increasing the functionality of the surgical instruments applied to circumstances already described.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of each claim of this application.

Throughout this specification the term “thromboembolectomy” is considered to relate to thrombectomy and/or embolectomy.

### SUMMARY OF THE INVENTION

According to a first aspect, the present invention consists in a thromboembolectomy device for performing a thromboembolectomy procedure in a bodily vessel, the device extending from a proximal end to a distal end. the device including:

a thromboembolectomy means capable of being moved relatively through the vessel and further capable of dislodging a thrombus or embolus from a wall of the vessel;

a capture means positioned distal the thromboembolectomy means, the capture means being movable at least between a first collapsed configuration and a second expanded configuration; and

an extractor means positioned proximal the thromboembolectomy means, the extractor means having a lumen, a first end and a second end wherein at least the second end is movable between a first collapsed state and a second expanded state;

wherein on dislodgment of the thrombus or embolus from a vessel wall by the thromboembolectomy means, the dislodged thrombus can be drawn relatively towards the extractor means for removal from the vessel and wherein further, any portions of thrombus that detach from the thrombus are retained by the capture means.

In a second aspect, the present invention consists in the capture means of the first aspect when used in the performance of a thromboembolectomy procedure in a bodily vessel.

In a third aspect, the present invention consists in the extractor means of the first aspect when used in the performance of a thromboembolectomy procedure in a bodily vessel.

In a fourth aspect, the present invention consists in a thromboembolectomy kit having its components enclosed in a sterile seal, the percutaneous thromboembolectomy kit at least including a percutaneous sheath; a catheter; and a thromboembolectomy device according to the first aspect of the invention.

In a fifth aspect, the present invention consists in a method for performing a percutaneous thromboembolectomy using a device according to the first aspect of the invention, the method including the steps of:

a) introducing a percutaneous sheath through a wall of a vessel in the body of a patient at an anatomical location in line with the vessel and proximal to a thrombus or embolus, such that the percutaneous sheath acts as means for accessing the lumen of the vessel;

b) introducing a guidewire through the lumen of the percutaneous sheath into the lumen of the vessel, so that it extends distally beyond the thrombus;

c) deploying a capture means according to the invention along the guidewire so that it reaches a position distal the thrombus;

d) expanding the capture means, or allowing it to expand, to an expanded configuration;

e) introducing a thromboembolectomy means along the guidewire so that the thromboembolectomy means reaches a position intermediate the thrombus and the capture means;

f) introducing an extractor means according to the invention along the guidewire so that its second end reaches a position proximal the thrombus;

g) expanding the second end of the extractor means, or allowing it to expand, to an expanded state;

h) withdrawing the thromboembolectomy means so that it removes the thrombus or embolus from the vessel wall and draws the removed thrombus or embolus through the lumen of the extractor means and out of the vessel to a location outside the body of the patient;

i) during the removal of the thromboembolectomy means as outlined in (h), allowing any portions of dislodged thrombus or embolus to be captured by the capture means;

j) withdrawing the guidewire and the capture means in an expanded or collapsed configuration, including any captured dislodged thrombus or embolus; and

k) withdrawing the extractor means and the percutaneous sheath.

The device according to each of the preceding aspects of the invention may be used in a range of surgical procedures involving the performance of open or percutaneous thromboembolectomies. The device may, however, be particularly suitable for use in performing percutaneous thromboembolectomies.

In a preferred embodiment of the invention each of the components of the device defined by the first aspect of the invention are used to carry out this invention.

The considerable advantages which arise as a result of using at least the capture means and the extractor means together, in combination with a conventional or specially manufactured thromboembolectomy means, will become clear in the foregoing detailed disclosure of preferred and alternative embodiments. However, either one or the other of the capture means and the extractor means in combination with a conventional or specially manufactured thromboembolectomy means may be used in performing percutaneous thromboembolectomies.

In a preferred embodiment of the invention, the capture means provides a mechanism for capturing portions of thrombus or embolus, dislodged from the surface of a thrombus or embolus while it is being removed from a vessel. In order to achieve this, the capture means must reach a position distal the thrombus or embolus and the thromboembolectomy means. Accordingly, the capture means is preferably introduced into the vessel prior to the introduction of the thromboembolectomy means.

Preferably, the capture means is capable of expanding such that it fills the entire cross-sectional diameter of the vessel in which it is positioned. This ensures that the capture means captures all dislodged thrombus or embolus.

The capture means may be moved between its first collapsed configuration and its second expanded configuration by a number of means including, but not limited to, manual alteration by the surgeon or alternatively without such manual alteration.

In a preferred embodiment wherein the surgeon manipulates the change in configuration of the capture means, the capture means may be formed of an appropriate solid surgical material including stainless steel, atraumatic plastic, or one of various alloys, with, for example, a construction resembling the mechanism of an umbrella. The capture means may further be made from a semisolid material or alternatively the capture means may be a balloon.

In one embodiment, where an umbrella mechanism is selected as the most appropriate means to achieve active manipulative control of the change of configuration of the capture means by the surgeon, the capture means is preferably secured to, or forms part of, the end of a guidewire such that the guidewire and capture means together form one component of the device of the present invention.

In a further embodiment, the device includes a mechanism to enable the surgeon to change the configuration of the capture means between the collapsed state and the expanded state from a position outside the patient. There are a number of alternatives for the formation of such a mechanism including, for example, having a reciprocating member which is adapted to engage the capture means and, upon reciprocation by the surgeon, cause the capture means to change configuration. Such a reciprocating member may take the form of an elongate wire which runs parallel to, and immediately alongside, the guidewire; or the guidewire itself may have a lumen inside which the reciprocating member is located. In the latter case, the reciprocating member may protrude from the lumen of the guidewire at its proximal end, such protrusion acting as a means for manipulation, and therefore, reciprocation of the reciprocating member, by the surgeon.

The capture means may be constructed from a first and, preferably, at least a second series of a plurality of spoke members, such that the first series of the plurality of spoke members are connected to. and extend radially from, a distal portion of the guidewire and each of the spoke members from the second series are connected to, and extend from, a central portion of each of the spoke members in the first series to a further connection with respective points of engagement on, the reciprocating member. Each of the connections between the guidewire and the spoke members from the first series, between the spoke members from the first series and the spoke members from the second series, and between the spoke members from the second series and the reciprocating member, are all freely moveable and may, for example, comprise individual pivot means.

In order to capture the greatest quantity of dislodged thromboemboli, the capture means preferably includes a large number of spoke members which, when the capture means is in the expanded configuration, span the entire cross-sectional diameter of the capture means with only very small gaps between the spoke members. It may, however, be desirable to secure a spanning means, formed of a material such as Dacron® or polytetrafluoroethylene (PTFE), to a surface of the first series of spoke members, such that the spanning means spans the entire cross-sectional diameter of the capture means. In such an embodiment, the spanning means may additionally, or alternatively, be formed from a material having a limited amount of diametric elasticity to ensure that it can be expanded into contact with the vessel wall, forming a seal between its outer circumference and the wall of the vessel and therefore further preventing escape of dislodged thrombus or embolus to locations beyond the capture means. In an alternative embodiment, the spanning means may take the configuration of a net or a semi-closed windsock.

The capture means may have the configuration of a cage structure. Such a structure may, for example, be in the shape of a wire cylinder having domed ends. In this case, like that of the umbrella mechanism, reciprocation of the reciprocating member results in a change in the capture means between the first collapsed configuration and the second expanded configuration.

The construction and material of the capture means may comprise an expansile foam. In this embodiment, manual manipulation by the surgeon to effect a change in the configuration of the capture means is not required. As the foam capture means is deployed along the guidewire and passes through the narrowed part of the vessel where the thrombus is situated, it will adopt a compressed configuration. Then, having passed the thrombus the foam capture means, by virtue of its construction, may take on an expanded configuration, thereby filling the entire cross-sectional diameter of the vessel. In this embodiment, it may further be necessary to incorporate a stopping member into the distal portion of the guidewire. Such a stopping member would prevent the capture means from extending distally beyond the end of the guidewire.

In a further embodiment, the capture means may be adapted such that it secures itself to the guidewire. Use of a material such as Nitinol™ is preferable, since a capture means formed of Nitinol™ will be capable of changing configuration from a compressed configuration to an expanded configuration when exposed to a change in temperature. Upon introduction into the body of a patient, the capture means undergoes an increase in temperature caused by its placement within the body of the patient. Consequently, the capture means, made from Nitinol™ may change its configuration from the first collapsed configuration to the second expanded configuration.

The capture means may have an orifice passing through its centre. The capture means may therefore engage the guidewire, and travel along it, because the guidewire can relatively slideably pass through the orifice of the capture means. When the capture means is in a compressed configuration, the orifice should be large enough so that the capture means can readily pass along the guidewire without significant friction between a rim of the orifice and an outer surface of the guidewire. However, the capture means of this embodiment should be constructed so that as its temperature increases (for example, following exposure to the patients body temperature), it changes to an expanded configuration and the size of its orifice gradually diminishes. As the orifice diminishes in size, there will be an increasing frictional force between a rim of the orifice and an outer surface of the guidewire leading to relatively greater difficulty in moving the capture means along the guidewire. This will continue until such time as the orifice becomes so small that any movement of the capture means is prevented.

In a further embodiment, while a change to one configuration of the capture means may be achieved without any manual manipulation, intervention by a surgeon may be required to achieve a change to a further configuration. In this embodiment, the capture means may be formed from a material having a “memory” of a particular configuration, such as an appropriate alloy with an ability to “memorise” physical configurations (eg Nitinol™. When manufactured, the capture means may be constructed in its expanded configuration. This would allow a surgeon to use a compressing means, such as a grasper or covering sheath, to compress the capture means to a collapsed configuration while introducing it into the vessel. Having moved the capture means beyond the narrowed portion of vessel where the thrombus or embolus is situated, the surgeon may release the grip of the compressing means or remove the covering sheath, thereby allowing the capture means to move into its expanded configuration. Alternatively, such a capture means could be manufactured in compliance with the umbrella mechanism described above, such that while the surgeon is required to manipulate the reciprocator member to cause the capture means to adopt a collapsed configuration, it would be capable of expanding by itself, by springing back into its “memorised” expanded configuration.

If the capture means is a balloon, the capture means may be constructed in the form of a balloon fixedly attached to a catheter. Such a construction would allow the capture means to be extended distally beyond the thromboembolectomy means, by slideably passing the catheter to which the capture means is fixedly attached through the other catheter to which the thromboembolectomy means is attached. Such a construction would obviate the need to introduce the capture means prior to the thromboembolectomy means during a thromboembolectomy procedure.

In a further embodiment, the extractor means may form a funnel-like structure which can cause the walls of the vessel proximal to the thrombus to be slightly dilated, and can compress and guide a thrombus which has been removed from the wall of a vessel through a previously introduced percutaneous sheath to a location external the body of the patient. In cases where both the extractor means and the capture means are used in the performance of a thromboembolectomy procedure, the extractor means may additionally operate to compress and guide any captured thromboemboli out through the percutaneous sheath.

Preferably, the second end of the extractor means may adopt, for example, a substantially frusto-conical shape. Alternatively, the second end may adopt any one of a number of different shapes which provide the extractor means with a substantially funnel-like structure. Further, a structure other than a funnel-like structure may be adopted.

The extractor means may be moved between the first collapsed state and the second expanded state by a number of means including manual alteration by a surgeon or, alternatively, without any manual manipulation. Alternatively, the extractor means may be moved between the first state and a second state by manipulation by a surgeon and moved to a further state without any manual manipulation.

The extractor means may be formed of an appropriate solid surgical material including stainless steel, atraumatic plastic, or one of various alloys. A particularly preferred material includes one of a series of alloys which have the capacity to “memorise” a particular structural configuration (eg Nitinol™).

Preferably, the extractor means includes a body with a tubular structure having its second end formed by a further structure having a substantially frusto-conical shape. Since the second end must be capable of collapsing and expanding between collapsed and expanded configurations, it may take the form of, for example: a plurality of compression members which radially extend from the most distal end of the tubular structure; a frustoconical spiral structure which extends from the most distal end of the tubular structure; or any other structure which can achieve the requisite change in configuration.

Preferably, the extractor means is manufactured such that the second end is in the expanded state, so that prior to inserting the extractor means, the surgeon will be required to manually compress the second end and introduce the extractor means into the percutaneous sheath. Extension of the extractor means beyond the distal end of the percutaneous sheath will then allow its second end to spring back to its memorised “expanded” state. In addition, having the second end of the extractor means spring back into its expanded state may also be achieved by securing the extractor means when it is in the desired position, and withdrawing the percutaneous sheath to expose the second end of the extractor means and release it from confinement within the percutaneous sheath.

The extractor means may be formed of Nitinol™ or any other “memory” alloy.

Where Nitinol™ is used, the extractor means may be pre-prepared such that its second end has a predetermined expanded shape which it may adopt upon heating (for example, following placement of the extractor means in the body of a patient). Once expanded, the second end will, provided that no deforming forces are applied thereto, retain and maintain that shape for at least the duration of the procedure. In this embodiment, it is preferred that the second end of the extractor means is comprised of a plurality of compression members which radially extend from the distal end of tubular body of the extractor means. Once the compression members have been exposed to an increase in temperature, they will each, when viewed from the side, preferably adopt a shape with a first outwardly curved and at least a second portion adjacent the first portion which is linear, such that: the outwardly curved portion extends from the distal end of the body of the extractor means and the linear portion extends from the distal end (opposite end) of the outwardly curved portion. Thus, according to this embodiment, when the second end of the extractor means has expanded to an expanded state, the linear portions of the compression members will be substantially parallel to the tubular body of the extractor means. In other words, the extractor means, in its entirety, is substantially cylindrical in shape with its second end having a cross-sectional diameter which tapers along the length of the outwardly curved portion of the compression members to a smaller cross-sectional diameter, which itself is the cross-sectional diameter of the body of the extractor means.

Preferably, when the second end of the extractor means is in an expanded state, its cross-sectional diameter will be larger than the cross-sectional diameter of the percutaneous sheath. Consequently, as the extractor means is withdrawn through the percutaneous sheath, the distal end of the percutaneous sheath will apply a deforming force to each of the outwardly curved portions of the compression members. This causes the distal ends of the linear portions of each compression member to move relatively toward one another, thereby-significantly decreasing the cross-sectional diameter of the lumen of the second end of the extractor means. This may enable substantial encapsulation of the thrombus or embolus, the thromboembolectomy means, and, in cases where the capture means is being used, the capture means along with any captured thrombus or embolus as well.

The extractor means and the percutaneous sheath may be manufactured as one component of the invention, wherein the extractor means is slideably secured within the lumen of the percutaneous sheath. Alternatively, the extractor means may be pre-compressed in its own sheath, which can be interlocked with the percutaneous sheath. In such a case, the extractor means may be slideably moved through its own sheath, through the percutaneous sheath, and into the vessel, after the two sheaths have been interlocked.

Where the extractor means is formed of the materials and construction which comply with the first or second possibilities already suggested, the extractor means should be manufactured accordingly, having particular regard to the disclosure of such combinations of materials and construction for the capture means (as described above).

Where the extractor means is formed of Nitinol™, all items may be withdrawn into the lumen of the extractor means, where, following a change in the second end of the latter to a collapsed configuration, they are all contained.

In a further embodiment wherein the extractor means and the capture means are both being used, it may be desirable to have the thromboembolectomy means wholly within the lumen of the extractor means, and the capture means at least partially contained within the extractor means, prior to the device being deployed. In this embodiment, the capture means would preferably be positioned at the distal end of the extractor means, and would, preferably, have a surface which provides safe passage of the extractor means into the vessel. Once inside the vessel, each of the capture means and the thromboembolectomy means may be deployed to their respective appropriate locations. At the conclusion of the procedure, the capture means may be withdrawn to its initial position within the extractor means. Consequently, the thromboembolectomy means, the thrombus, embolus and any captured thromboemboli would be encapsulated by the extractor means and capture means, prior to their withdrawal from the body of a patient.

In an alternative embodiment of the invention, whether the capture means and the extractor means are both employed or whether only one of them is employed, it may be desirable to additionally use a catheter with a plurality of perforations in a wall at its distal end as part of the apparatus for carrying out this invention. Such a catheter, may be introduced into the vessel over the guidewire as an independent component of the invention. It may also be introduced in combination with a thromboembolectomy means, as some conventional thromboembolectomy means have a catheter as an integral component of their construction.

The perforations provide a means for delivering thrombolytic substances to the area in the vessel defined by the thrombus, the capture means and the vessel walls. Delivery of the thrombolytic substances will further ensure that the thrombus, embolus and the dislodged thromboemboli are completely removed.

In a further preferred embodiment, the device may be easily adapted for a variety of surgical procedures. For example, where a thrombus or embolus is to be removed from one of the femoral arteries, (ie in a region which is difficult to access on the ipsilateral side), a guidewire may be passed through the femoral artery on the contralateral side of the patient, and the individual components of the device deployed along the guidewire, around the bifurcation of the aorta and into the vessel containing the thrombus or embolus. Hence, use of a device according to this invention is not limited to removing a thrombus or embolus from the ipsilateral side of the patient only.

### BRIEF DESCRIPTION OF THE DRAWINGS

By way of example, preferred embodiments of the invention are described with reference to the accompanying drawings in which:

FIG. 1 is a schematic diagram of a device according to this invention, as introduced into a vessel in the body of a patient with all of its components in their respective positions prior to removal of a thrombus;

FIG. 2a is a perspective diagram of a capture means according to one embodiment of the invention which is an integral component of a guidewire;

FIG. 2b is a perspective diagram of a capture means according to another different embodiment of the invention to that illustrated in FIG. 2a which has engaged a guidewire;

FIG. 2c is another perspective diagram of a capture means according to one embodiment of the invention which is an integral component of a guidewire;

FIG. 2d is a cross-sectional diagram illustrating one further preferred embodiment wherein the capture means is a balloon.

FIG. 3a is a perspective diagram of an extractor means according to one embodiment of the invention;

FIG. 3b is a perspective diagram of an extractor means according to another different embodiment of the invention to that illustrated in FIG. 3a;

FIG. 3c is a perspective diagram of an extractor means according to another embodiment of the invention;

FIG. 4a is schematic diagram illustrating an example of the physical relationship between an extractor means and a percutaneous sheath when the distal portion of the extractor means is being maintained in the compressed state by the confines of the percutaneous sheath;

FIG. 4b is a schematic diagram illustrating an example of the physical relationship between an extractor means and a percutaneous sheath when the distal portion of the extractor means has expanded to an expanded state having been released from the confines of the percutaneous sheath;

FIG. 4c is schematic diagram illustrating an example of the physical relationship between an extractor means formed of Nitinol™ and a percutaneous sheath when the distal portion of the extractor means has expanded to an expanded state;

FIG. 4d is schematic diagram of FIG. 4c illustrating the change in relationship of the distal ends of the compression members of an extractor means, when the latter is being withdrawn through the percutaneous sheath;

FIG. 5a is a cut-away perspective diagram illustrating one preferred embodiment of the invention, wherein prior to deployment, the thromboembolectomy means is wholly within the lumen of the extractor means, and the capture means is at least partially within the lumen of the extractor means;

FIG. 5b is a cut-away perspective diagram of FIG. 5a illustrating one preferred embodiment of the invention, wherein following removal of the thrombus, the thromboembolectomy means and capture means are withdrawn back into the lumen of the extractor means before all of these components are removed from the body of the patient; and

FIG. 6 is a diagrammatic partially cut-away ventral view of a patient illustrating how the invention can be used to remove a thrombus from a vessel on the contralateral side of the patient from which surgical access has been gained.

### PREFERRED MODE OF CARRYING OUT THE INVENTION

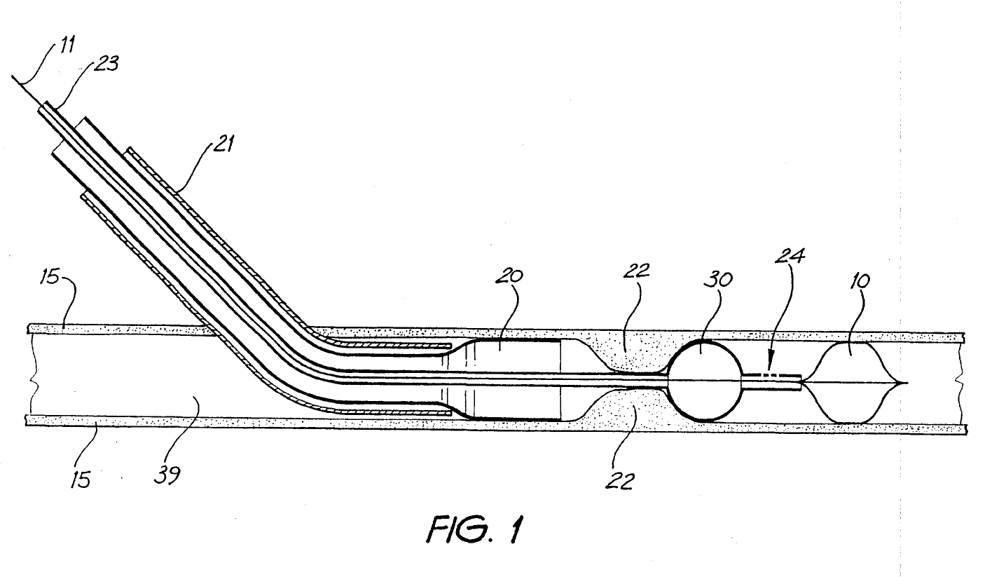

A capture means 10 and an extractor means 20 are adapted for use with a conventional or specially manufactured thromboembolectomy means 30 in the performance of a percutaneous thromboembolectomy (see FIG. 1).

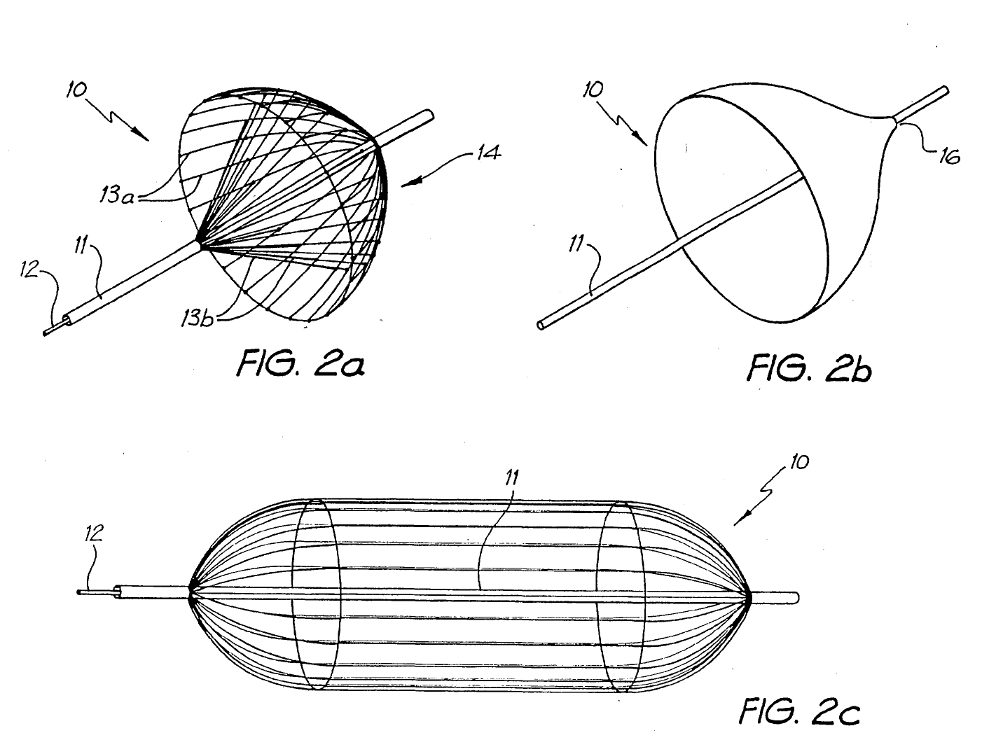

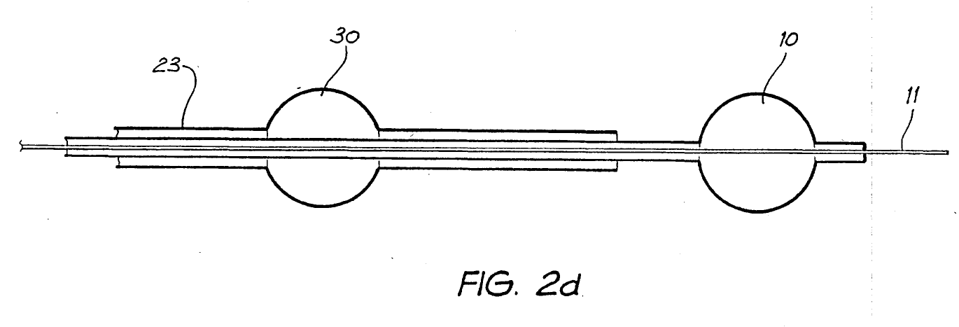

The capture means 10 may be manufactured using a variety of combinations of materials and construction. In a preferred embodiment, wherein the surgeon manipulates the change in configuration of the capture means 10 between collapsed and expanded configurations, the capture means 10 may be formed of an appropriate solid surgical material including stainless steel, atraumatic plastic, or one of various alloys, with, for example, a construction resembling the mechanism of an umbrella (see FIG. 2a). Alternatively, the capture means 10 may be a balloon.

Where an umbrella mechanism is selected as the most appropriate means to achieve active manipulative control of the change of configuration for the capture means 10 by the surgeon, the capture means 10 should be secured to, or form part of, the end of a guidewire 11, such that the guidewire 11 and capture means 10 form one component of the invention.

In such an embodiment, a mechanism allowing the surgeon to change the configuration of the capture means 10 between collapsed and expanded states from a position outside the patient will also form part of the capture means 10 and guidewire 11 component of the invention. There are a number of alternatives for the formation of such a mechanism including, for example, having a reciprocating member 12 which is adapted to engage the capture means and, upon reciprocation by the surgeon, cause the capture means 10 to change configuration. Such a reciprocating member 12 may take the form of an elongate wire which runs parallel to, and immediately alongside, the guidewire 11; or, the guidewire 11 itself may have a lumen inside which the reciprocating member is located (see FIG. 2a). In the latter case, the reciprocating member 12 may protrude from the lumen of the guidewire 11 at its proximal end, such protrusion acting as a means for manipulation, and therefore, reciprocation of the reciprocating member 12, by the surgeon.

In this embodiment, the capture means 10 itself is constructed by a first and a second series of a plurality of spoke members 13a and 13b respectively, such that: the first series of a plurality of spoke members 13a are connected to, and extend radially from, a distal portion of the guidewire; and each of the spoke members from the second series 13b are connected to, and extend from, a central portion of each of the spoke members in the first series 13a to a further connection with respective points of engagement on the reciprocating member 12. Each of the connections between the guidewire 11 and the spoke members 13a from the first series, between the spoke members from the first series 13a and the spoke members 13b from the second series, and between the spoke members 13b from the second series and the reciprocating member 12, are all freely moveable and may, for example, comprise individual pivot means.

It may further be desirable to secure a spanning means 14, formed of a material such as Dacron® or polytetrafluoroethylene (PTFE), to a surface of the first series of spoke members 13a, such that the spanning means 14 spans the entire cross-sectional diameter of the capture means 10. In such embodiments, the spanning means 14 may additionally, or alternatively, be formed from a material having a limited amount of diametric elasticity to ensure that it can be expanded into contact with the wall 15 of the vessel 39, forming a seal between its outer circumference and the wall 15 of the vessel 39 and therefore further preventing escape of dislodged thromboemboli (not shown) to locations beyond the capture means 10. In alternative embodiments, the spanning means 14 may take the configuration of a net or a semi-closed windsock.

In addition, as mentioned in the description of the invention, alternative embodiments of the capture means 10 which use a similar functional mechanism to that of the umbrella configuration (see FIG. 2a), disclose that the capture means 10 has a cage structure. Such a structure may, for example, substantially be in the shape of a wire cylinder having domed ends (see FIG. 2c). In this case, like that of the umbrella mechanism (FIG. 2a), reciprocation of the reciprocating member 12 results in a change in the configuration of the capture means 10 between a collapsed and expanded configuration.

It is also possible, however, in embodiments of the invention wherein a change in configuration of the capture means 10 will occur without manipulation by a surgeon, for the materials and construction of the capture means 10 to be selected such that the capture means 10 secures itself to the guidewire 11 (see FIG. 2b). In such embodiments, use of a material such as Nitinol™ is preferable, since a capture means 10 formed of Nitinol™ will be capable of changing configuration from a compressed configuration to an expanded configuration when exposed to a change in temperature: upon being introduced into the body of a patient, the capture means 10 will undergo an increase in temperature caused by its placement within the body of the patient, and will consequently change its configuration from an initial collapsed configuration to an expanded configuration.

In such a case, the capture means 10 will have an orifice 16 passing through its centre. The capture means 10 can therefore engage the guidewire 11, and travel along it, because the guidewire 11 can relatively slideably pass through the orifice 16 of the capture means 10. When the capture means 10 is in a compressed configuration, the orifice 16 should be large enough so that the capture means 10 can pass along the guidewire 11 significant friction between a rim of the orifice 16 and an outer surface of the guidewire 11. However, the capture means 10 of this embodiment should be constructed so that as its temperature increases (after, for example, having been exposed to the patient's body temperature), it changes to an expanded configuration and the size of its orifice 16 gradually diminishes. As the orifice 16 diminishes in size, there will be an increasing frictional force between a rim of the orifice 16 and an outer surface of the guidewire 11 thereby increasing the difficulty of movement of the capture means 10 along the guidewire ii. This will continue until such time as the orifice 16 becomes so small that any movement of the capture means 10 is prevented.

The capture means 10 may comprise a balloon (see FIG. 2d) fixedly attached to a catheter. Such a construction would allow the capture means 10 to be extended distally beyond the thromboembolectomy means 30, by slideably passing the catheter to which the capture means 10 is fixedly attached through the catheter to which the thromboembolectomy means 30 is attached. Such a construction would obviate the need to introduce the capture means 10 prior to the thromboembolectomy means 30 during a thromboembolectomy procedure.

Like the capture means 10, the extractor means 20 may be manufactured of a variety of combinations of materials and construction. In a preferred embodiment, however, the extractor means 20 may be formed of an appropriate solid surgical material including stainless steel, atraumatic plastic, or one of various alloys. A particularly preferred material for such embodiments, however, is one of a series of alloys which have the capacity to “memorise” a particular structural configuration; or Nitinol™ because of its ability to change configuration following being subjected to a change in temperature.

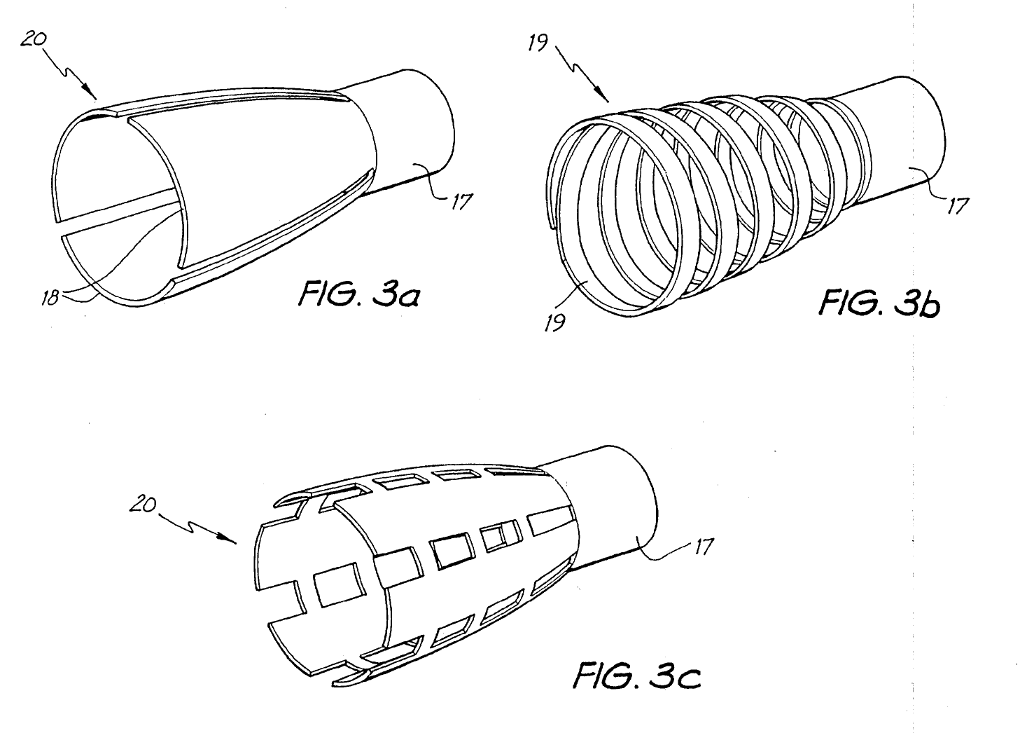

An extractor means 20 according to this invention comprises a body with a tubular structure 17 having its second end formed by a further structure having a substantially frusto-conical shape. Since the second end must be capable of collapsing and expanding between collapsed and expanded states, it may take the form of, for example, a plurality of compression members 18 which radially extend from the most distal aspect of the tubular structure (see FIG. 3a), a frusto-conical spiral structure 19 which also extends from the most distal end of the tubular structure (see FIG. 3b) or any other structure which can achieve the requisite change in state (see, for example, FIG. 3c).

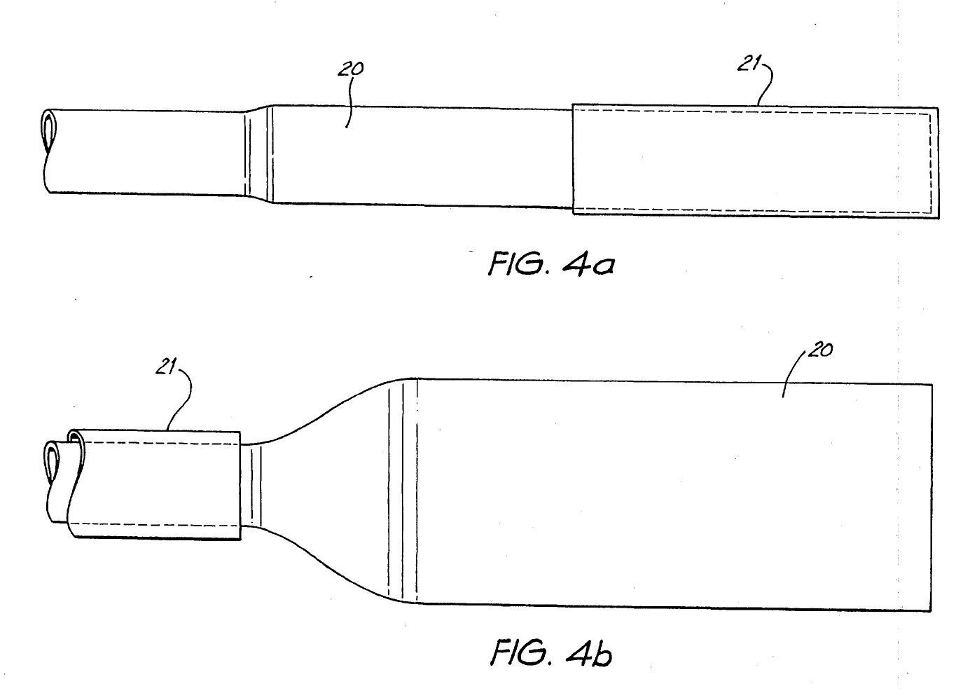

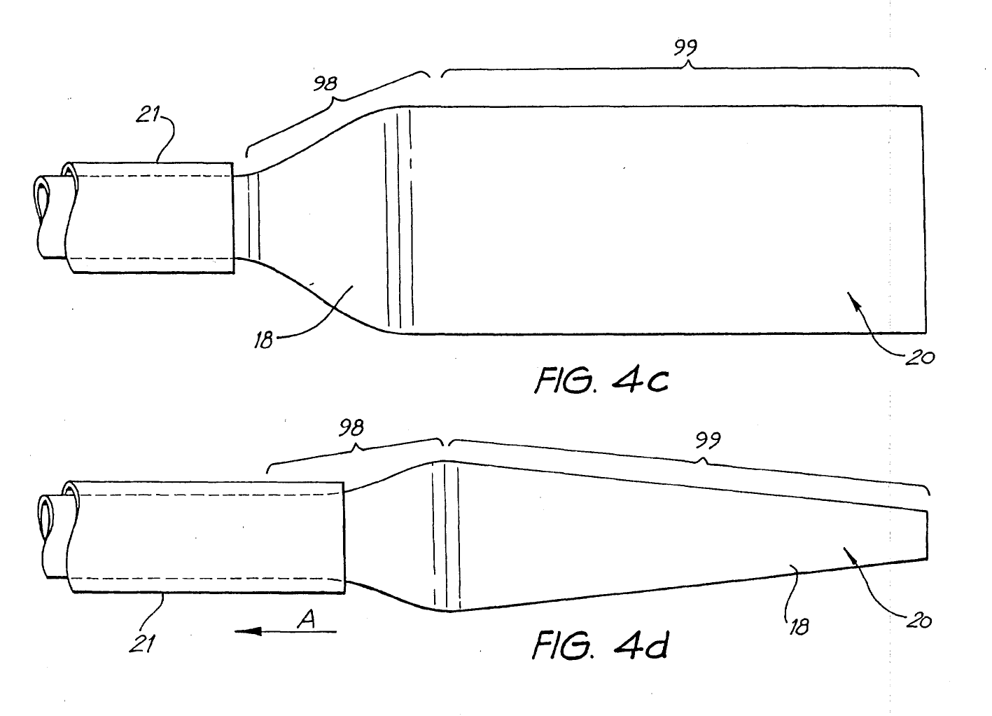

When manufactured according to this embodiment, an extractor means 20 should initially have its second end in the expanded state, so that prior to inserting the extractor means 20, the surgeon will be required to manually compress the second end and introduce the extractor means 20 into the percutaneous sheath 21. As shown in FIG. 4b, relative extension of the extractor means 20 beyond the distal end of the percutaneous sheath 21 will then allow its second end to spring back to its memorised “expanded” state. In addition, it is noteworthy that having the second end of the extractor means 20 spring back into its expanded state can also be achieved by securing the extractor means 20 when it is in the appropriate position, and withdrawing the percutaneous sheath 21 to expose the distal portion of the extractor means 20 and release it from confinement within the percutaneous sheath 21 (also see FIG. 4b).

In further preferred embodiments of the invention in which Nitinol™ is used, the extractor means 20 may be pre-prepared such this its second end has a predetermined expanded shape which it may adopt upon heating (for example, following placement of the extractor means 20 in the body of a patient). Once expanded, the second end will, provided that no deforming forces are applied thereto, retain and maintain that shape for at least the duration of the procedure. According to these embodiments, it is preferable that the second end of the extractor means 20 is comprised of a plurality of compression members 18 which radially extend from the distal end of the body 17 (as described above; see also FIG. 3a). While not depicted, the compression members may be separated by an area of webbing or similar material. Once the compression members 18 have been exposed to an increase in temperature, they will each, when viewed from the side, preferably adopt a shape with an outwardly curved first portion 98 and at least a second portion 99 adjacent the first portion 98 which is linear, such that: the outwardly curved portion 98 extends from the distal end of the body 17 of the extractor means 20; and the linear portion 99 extends from portion 98. Thus, according to this embodiment, when the second end of the extractor means 20 has expanded to an expanded state, and is not affected by deforming forces, the linear portions 99 of the compression members 18-will be substantially parallel to the tubular body 17 of the extractor means 20. In other words, the extractor means 20, in its entirety, is substantially cylindrical in shape with its second end having a cross-sectional diameter which tapers over the length of portion 98 of the compression members 18 to a smaller cross-sectional diameter, which itself is the cross-sectional diameter of the body 17 of the extractor means 20.

As illustrated in FIG. 4c, when the second end of the extractor means 20 of such embodiments is in an expanded state, its cross-sectional diameter will be larger than the cross-sectional diameter of the percutaneous sheath 21. Consequently, as the extractor means 20 is withdrawn through the percutaneous sheath 21 as indicated by arrow A in FIG. 4d, the distal end of the percutaneous sheath will apply a deforming force to each of the hyperbolic portions 98 of the compression members 18. This causes the distal ends of the linear portions 99 of each compression member 18 to move relatively toward one another, thereby significantly decreasing the cross-sectional diameter of the lumen of the second end of the extractor mean 20.

These embodiments may be particularly beneficial, since they provide a means to substantially encapsulate the thrombus 22, the thromboembolectomy means 30, and, in cases where the capture means 10 is being used, the capture means 10 along with any captured thromboemboli (not shown) as well.

The extractor means 20 depicted in the drawings are elongate in structure such that they extend towards the distal end of the body 17. When in use, the elongate body 17 extends substantially parallel to a vessel wall, both in compressed and expanded states. Upon expansion of the extractor member, a portion of the elongate body 17 abuts with the vessel wall as shown in FIG. 6.

In some of the embodiments discussed above, there are two ways in which the extractor means 20 may be preferably manufactured: first, it may be appropriate and/or more suitable to manufacture the extractor means 20 and the percutaneous sheath 21 as one component of the invention, wherein the extractor means 20 is slideably secured within the lumen of the percutaneous sheath 21 (see, for example, FIG. 4a); or, the extractor means 20 may be pre-compressed in its own sheath (not shown), which can be interlocked with the percutaneous sheath 21. In such cases as the latter option for manufacture is selected, the extractor means 20 may be slideably moved through its own sheath (not shown), through the percutaneous sheath 21, and into the vessel 39, after the extractor member's sheath (not shown) and the percutaneous sheath 21 have been interlocked.

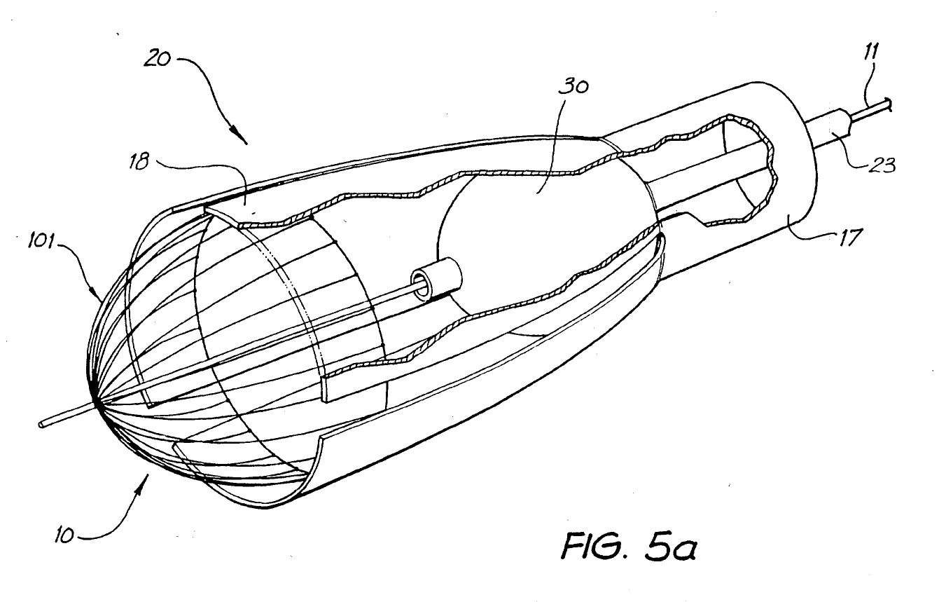

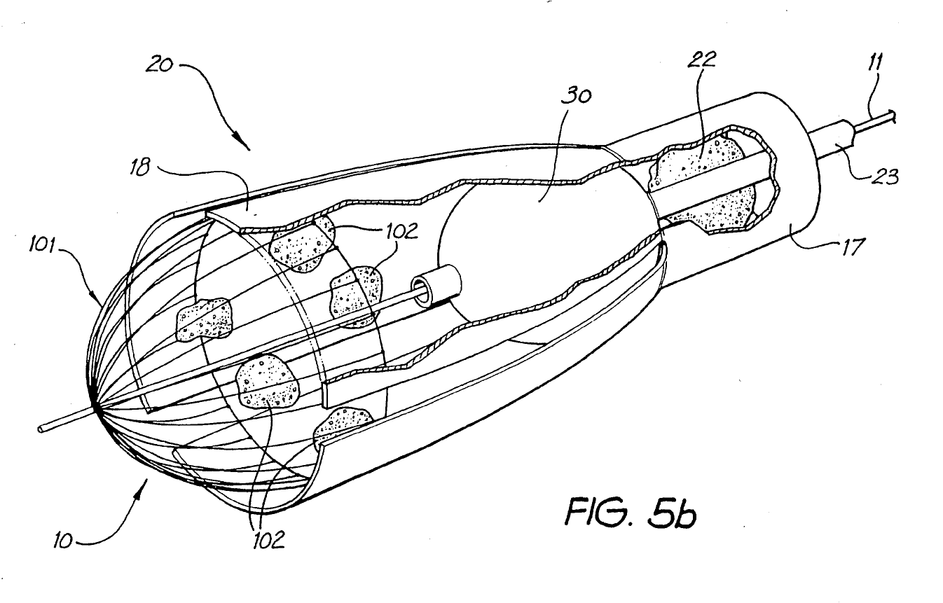

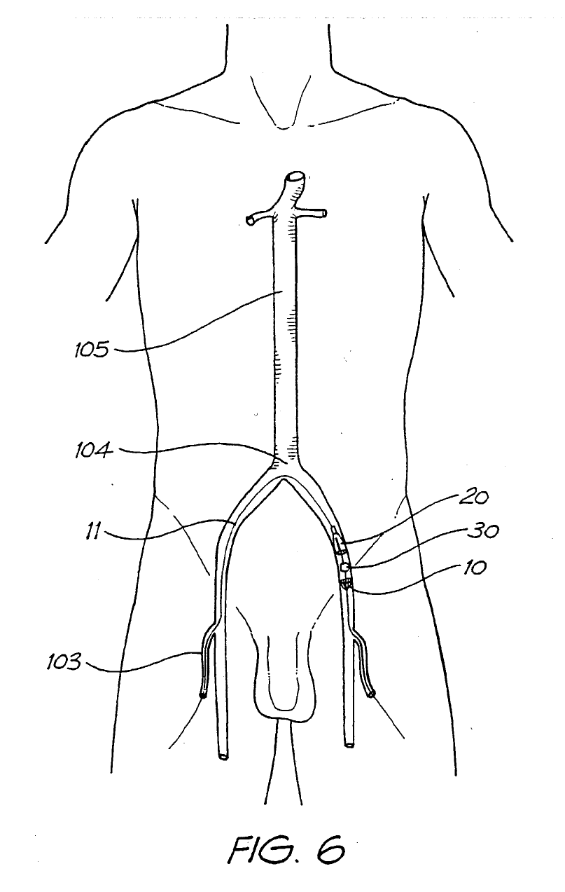

When the extractor means 20 and the capture means 10 are both being used, it may be desirable to have the thromboembolectomy means 30 wholly within the lumen of the extractor means 20, and the capture means 10 at least partially within the lumen of the extractor means 20, prior to the devices being deployed. As shown in FIG. 5a the capture means 10 would be at the most distal end of the extractor means 20, and would, preferably, have a surface 101 which provides safe passage of the extractor means 20 into the vessel 39. Once inside the vessel 39, each of the capture means 10 and thromboembolectomy means 30 can be deployed to their respective appropriate locations. At the conclusion of the procedure, the capture means 10 would then be withdrawn to its initial position within the extractor means 20. Consequently, as illustrated by FIG. 5b, the thromboembolectomy means 30, the thrombus 22 and any captured thromboemboli 102 would be encapsulated by the extractor means 20 and capture means 10, prior to their withdrawal from the body of a patient.

Using any of the embodiments of the capture means 10 and any of the embodiments of the extractor means 20, the preferred method for carrying out a percutaneous thromboembolectomy according to this invention includes the following steps:

a) introducing a percutaneous sheath 21 through a wall 15 of a vessel 39 in the body of a patient at an anatomical location in line with the vessel 39 and proximal to a thrombus 22, such that the percutaneous sheath 21 acts as means for accessing the lumen of the vessel 39;

b) introducing a guidewire 11 through the lumen of the percutaneous sheath 21 into the lumen of the vessel 39, so that it extends distally beyond the thrombus 22;

c) deploying the capture means 10 in a compressed configuration along the guidewire 11 so that it reaches a position distal the thrombus 22;

d) expanding the capture means 10, or allowing it to expand, to an expanded configuration;

e) introducing a conventional or specially manufactured thromboembolectomy means 30 along the guidewire 11 so that the thromboembolectomy means 30 reaches a position intermediate the thrombus 22 and the capture means 10;

f) introducing the extractor means 20 with its second end in a collapsed state along the guidewire 11 so that its second end reaches a position proximal the thrombus 22;

g) expanding the second end of the extractor means 20, or allowing it to expand, to an expanded state;

h) withdrawing the thromboembolectomy means 30-50 that it removes the thrombus 22 from the wall 15 of the vessel 39 and draws the removed thrombus 22 through the lumen of the extractor means 20 and out of the vessel 39 to a location outside the body of the patient;

i) during the removal of the thromboembolectomy means 30 as outlined in (h), allowing any portions of dislodged thrombus, or thromboemboli (not shown), to be captured by the capture means 10;

j) withdrawing the guidewire 11 and the capture means 10 in an expanded or collapsed configuration, including any captured dislodged thrombus, or thromboemboli (not shown); and

k) withdrawing the extractor means 20 and the percutaneous sheath 21.

In alternative embodiments of the invention, whether the capture means 10 and the extractor means 20 are both employed or whether only one of them is employed, it may be desirable to additionally use a catheter 23 with a plurality of perforations 24 in a wall at its distal end as part of the apparatus for carrying out this invention. Such a catheter 23, may be introduced into the vessel 39 over the guidewire 11 as an independent component of the invention. It may also be introduced in combination with an thromboembolectomy means 30, as some conventional thromboembolectomy means 30 have a catheter 23 as an integral component of their construction.

The perforations 24 provide a means for delivering thrombolytic substances to the area in the vessel 39 defined by the thrombus 22, the capture means 10 and the walls 15 of the vessel 39. Delivery of the thrombolytic substances will be yet another means to ensure that the thrombus 22 and the dislodged thromboemboli (not shown) are completely removed.

The device according to this invention can easily be adapted to deal with a variety of surgical circumstances (see FIG. 6). For example, where a thrombus 22 is to be removed from one of the femoral arteries, (ie. from a region which is difficult to access on the ipsilateral side), a guidewire can be passed through the femoral artery 103 on the contralateral side of the patient, and the individual components of the device can be deployed along the guidewire, around the bifurcation 104 of the aorta 105 and into the vessel 39 containing the thrombus 22. Hence, use of a device according to this invention is not limited to removing a thrombus 22 from the ipsilateral side of the patient only.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A thromboembolectomy device for performing a thromboembolectomy procedure in a bodily vessel, the device extending from a proximal end to a distal end, the device including:
a thromboembolectomy means capable of being moved relatively through the vessel and further capable of dislodging a thrombus or embolus from a wall of the vessel;
a capture means positioned distal the thromboembolectomy means, the capture means being movable at least between a first collapsed configuration and a second expanded configuration; and
an extractor means positioned proximal the thromboembolectomy means, the extractor means having a lumen, a first end and a second end wherein at least the second end is movable between a first collapsed state and a second expanded state;
wherein on dislodgment of a thrombus or embolus from a vessel wall by the thromboembolectomy means, the dislodged thrombus can be drawn relatively towards the extractor means for removal from the vessel and wherein further, any portions of thrombus that detach from the thrombus are retained by the capture means.

2. The device of claim 1 wherein the capture means resembles the shape and has the mechanism of an umbrella.

3. The device of claim 1 or claim 2 wherein the capture means is secured to, or forms part of, the end of a guidewire.

4. The device of any one of the preceding claims further including a control mechanism to enable a surgeon to move the capture means between the first collapsed configuration and the second expanded configuration.

5. The device of claim 4 wherein the control mechanism includes a reciprocating member which is adapted to engage the capture means and, upon reciprocation by the surgeon, cause the capture means to move between the first collapsed configuration and the second expanded configuration.

6. The device of claim 5 wherein the reciprocating member includes an elongate wire which runs parallel and alongside the guidewire.

7. The device of claim 5 or claim 6 wherein the capture means includes a first and at least a second series of a plurality of spoke members, such that the first series of the plurality of spoke members are connected to, and extend radially from, a distal portion of the guidewire and each of the spoke members from the second series are connected to, and extend from, a central portion of each of the spoke members in the first series to a further connection with respective points of engagement on the reciprocating member.

8. The device of claim 7 wherein each of the connections between the guidewire and the spoke members from the first series, between the spoke members from the first series and the spoke members from the second series, and between the spoke members from the second series and the reciprocating member, are all freely moveable.

9. The device of claim 7 or claim 8 further including a spanning means secured to a surface of the first series of spoke members, such that the spanning means spans the entire cross-sectional diameter of the capture means.

10. The device of claim 9 wherein the spanning means is made from a material selected from the group consisting of Dacron® or polytetrafluoroethylene (PTFE).

11. The device of any one of the preceding claims wherein the capture means is made from a material selected from the group consisting of stainless steel, atraumatic plastic, or an alloy.

12. The device of any one of claims 1 to 10 wherein the capture means is made from an expansile foam.

13. The device of any one of claims 1 to 11 wherein the capture means is made from a “shape memory” material.

14. The device of claim 13 wherein the capture means is made from Nitinol™.

15. The device of claim 3 wherein the capture means has a central orifice for passage of the guidewire therethrough.

16. The device of claim 1 wherein the capture means is a balloon.

17. The device of claim 16 wherein the balloon is fixedly attached to a catheter.

18. The device of any one of the preceding claims wherein the entire extractor means is capable of moving between a first collapsed state and a second expanded state.

19. The device of any one of the preceding claims wherein the extractor means is substantially funnel-like in structure.

20. The device of claim 19 wherein a region adjacent the second end of the extractor means is substantially frusto-conical in shape.

21. The device of any one of the preceding claims wherein the extractor means is formed from a surgical material selected from the group consisting of stainless steel, atraumatic(plastic, or an alloy.

22. The device of any one of the preceding claims wherein the extractor means is made from a “shape memory” material.

23. The device of claim 22 wherein the extractor means is made from Nitinol™.

24. The device of claim 1 wherein a region adjacent the second end of the extractor means includes a plurality of compression members which radially extend from a main tubular body.

25. The device of any one of the preceding claims wherein the extractor means is capable of substantially encapsulating the thrombus, the thromboembolectomy means and the capture means.

26. The device of claim 1 further including a catheter having a body extending from a proximal to a distal end, the catheter having a plurality of perforations in a wall at its distal end.

27. The device of claim 26 wherein the perforations in the wall of the catheter provide a means for delivering thrombolytic substances to an area in the vessel.

28. A method for performing a percutaneous thromboembolectomy using the thromboembolectomy device of claim 1, the method including the steps of:
a) introducing a percutaneous sheath through a wall of a vessel in the body of a patient at an anatomical location in line with the vessel and proximal to a thrombus, such that the percutaneous sheath acts as means for accessing the lumen of the vessel;
b) introducing a guidewire through the lumen of the percutaneous sheath into the lumen of the vessel, so that it extends distally beyond the thrombus;
c) deploying a capture means according to the invention along the guidewire so that it reaches a position distal the thrombus;
d) expanding the capture means, or allowing it to expand, to an expanded configuration;
e) introducing an thromboembolectomy means along the guidewire so that the thromboembolectomy means reaches a position intermediate the thrombus and the capture means;
f introducing an extractor means according to the invention along the guidewire so that its second end reaches a position proximal the thrombus;
g) expanding the second end of the extractor means, or allowing it to expand, to an expanded state;
h) withdrawing the thromboembolectomy means so that it removes the thrombus from the vessel wall and draws the removed thrombus through the lumen of the extractor means and out of the vessel to a location outside the body of the patient;
i) during the removal of the thromboembolectomy means as outlined in (h), allowing any portions of dislodged thrombus, or thromboemboli, to be captured by the capture means;
j) withdrawing the guidewire and the capture means in an expanded or collapsed configuration, including any captured dislodged thrombus, or thromboemboli; and
k) withdrawing the extractor means and the percutaneous sheath.

29. A thromboembolectomy device when used for performing a thromboembolectomy procedure in a bodily vessel, the device including the capture means of claim 1 for use in the performance of a thromboembolectomy procedure in a bodily vessel.

30. A thromboembolectomy device when used for performing a thromboembolectomy procedure in a bodily vessel, the device including the extractor means of claim 1 for use in the performance of a thromboembolectomy procedure in a bodily vessel.

31. A thromboembolectomy kit having its components enclosed in a sterile seal, the thromboembolectomy kit at least including: a percutaneous sheath; a catheter; and a thromboembolectomy device according to claim 1.
